(19) United States
(12) Patent Application Publication
(10) Pub. No.: US 2026/0301472 A1
(43) Pub. Date: Oct. 1, 2026

(54) BIOMETRIC VERIFICATION

(71) Applicant: Candour Oy, Oulu (FI)

(72) Inventors: Jukka KOMULAINEN, Oulu (FI); Harri KOSKIMÄKI, Oulu (FI); Olli SILVÉN, Oulu (FI)

(21) Appl. No.: 19/013,868

(22) PCT Filed: Jun. 20, 2023
(86) PCT No.: PCT/FI2023/050373

§ 371 Date: Jan. 8, 2025

(30) Foreign Application Priority Data
Jul. 8, 2022 (FI) 20225646

(51) Int. Cl. G06V 40/40; A61B 5/024; A61B 5/103; A61B 5/11; A61B 5/117; G06V 40/10; G06V 40/16

(52) U.S. Cl. CPC G06V 40/45; A61B 5/02416; A61B 5/1032; A61B 5/1102; A61B 5/117; G06V 40/11; G06V 40/162; A61B 2562/06

(57) ABSTRACT

A method, an apparatus, and a computer program product for biometric verification are disclosed. The method comprises: obtaining a first biosignal indicative of a heart rate of a subject, wherein the first biosignal has been measured by a first sensor; obtaining a second biosignal indicative of the heart rate of the subject, wherein the second biosignal has been measured by a second sensor that is different from the first sensor, and wherein the first and second biosignals have been measured at the same time; determining a delay between the first and second biosignals; and verifying liveness of the subject based on the determined delay.

### TECHNICAL FIELD

The present solution generally relates to a method, an apparatus, and a computer program product for biometric verification.

### BACKGROUND

Face-based identity verification methods have become a common replacement for passwords and can supplement other biometric identification methods such as fingerprints. The identity verification is often done using a smartphone by capturing selfie pictures or a video of the user.

All biometric methods can be subject to presentation attacks, also known as spoofing attacks. Anti-spoofing techniques are incorporated into identity verification systems to raise the technical difficulty and cost of misuse by impostors.

Common techniques for spoofing face-based identity verification methods include deepfake pictures and videos, and impostors wearing masks. Various liveness detection methods can be employed to combat this type of spoofing. For instance, the user may be requested to perform an action such as turning their head. However, this approach is vulnerable to deepfake video generated in real-time based on the impostor's respective actions. Another video-based liveness detection approach is to detect the heart rate of the user from minute changes of skin color on the face. This is effective against masks and still pictures, but may fail against advanced real-time deepfake videos that imitate the color changes associated with the heart rate.

### SUMMARY OF THE INVENTION

The scope of protection sought for various embodiments of the invention is set out by the independent claims. Various embodiments are disclosed in the dependent claims. Although various aspects of the embodiments are set out in the independent claims, other aspects comprise other combinations of features from the described embodiments and/or the dependent claims with the features of the independent claims, and not solely the combinations explicitly set out in the claims.

A method for biometric verification comprises obtaining a first biosignal indicative of a heart rate of a subject, wherein the first biosignal has been measured by a first sensor; obtaining a second biosignal indicative of the heart rate of the subject, wherein the second biosignal has been measured by a second sensor that is different from the first sensor, and wherein the first and second biosignals have been measured at the same time; determining a delay between the first and second biosignals; and verifying liveness of the subject based on the determined delay.

The method may be a computer-implemented method.

The method may further comprise outputting a pass result or a failure result of the verification.

Verifying the liveness of the subject may comprise comparing the determined delay to an expected delay, and, if the determined delay matches the expected delay, passing liveness verification of the subject, and otherwise failing the liveness verification of the subject.

The method may further comprise determining a delay signal comprising a plurality of delay values between the first and second biosignals, and comparing each value of the delay signal to the expected delay, and, if all values of the delay signal match the expected delay, passing liveness verification of the subject, and otherwise failing the liveness verification of the subject.

Each delay value of the delay signal may correspond to a delay of a single heartbeat between the first biosignal and the second biosignal.

The first sensor may be of a different type than the second sensor.

The first biosignal may be of a different type than the second biosignal.

The first biosignal may comprise a remote photoplethysmography signal, and the second biosignal may comprise a ballistocardiography signal.

The first biosignal may comprise a remote photoplethysmography signal comprising color distributions of one or more skin regions of the face of the subject.

The first biosignal and the second biosignal may be measured from different body parts of the subject.

The first biosignal may be measured from a first body part having a first distance to the heart of the subject, and the second biosignal may measured from a second body part having a second distance to the heart of the subject, wherein the first and second distances are different.

The first biosignal may be measured from the face of the subject, and the second biosignal may be measured from the hand of the subject.

Obtaining the first biosignal may comprise measuring the first biosignal using the first sensor.

The first sensor may comprise a camera configured to measure image data indicative of the heart rate of the subject.

Obtaining the first biosignal may comprise acquiring a plurality of color image data frames depicting the face of the subject; detecting color content indicative of the heart rate of the subject in the plurality of color image data frames; and generating the first biosignal based on the detected color content.

Detecting the color content may comprise identifying one or more skin regions in each of the plurality of color image data frames; extracting a skin region data set from each of the one or more identified skin regions in each of the plurality of color image data frames; and detecting the color content of each extracted skin region data set.

The method may further comprise computing a plurality of color distributions, each color distribution being computed on the basis of one of the plurality of skin region data sets; and detecting the color content of each extracted skin region data set based on the color distribution computed on the basis of said skin region data set.

Obtaining the second biosignal may comprise measuring the second biosignal using the second sensor.

The second sensor may comprise a movement sensor configured to measure movement sensor data indicative of the heart rate of the subject.

The second biosignal may comprise a ballistocardiography signal based on the movement sensor data.

The method may further comprise measuring the first and second biosignals using the same device.

An apparatus is configured to perform the method.

The apparatus may comprise at least one processor, at least one memory including computer program code, the at least one memory and the computer program code configured to, with the at least one processor, cause the apparatus to perform the method.

The apparatus may further comprise the first sensor for measuring the first biosignal and the second sensor for measuring the second biosignal.

The apparatus may be a handheld apparatus or a wearable apparatus.

A computer program product comprises computer program code configured to, when executed by at least one processor, cause an apparatus to perform the method.

The computer program product may be a embodied on a non-transitory computer-readable medium.

### BRIEF DESCRIPTION OF THE DRAWINGS

FIG. 1 illustrates an example biometric system;

FIG. 2 is a schematic diagram depicting an embodiment of an apparatus according to the invention;

FIG. 3 is a flow chart illustrating a method according to the invention;

FIG. 4 shows a use case of a second embodiment of an apparatus according to the invention;

FIG. 5 depicts a spoofing attempt; and

FIG. 6A to FIG. 6D show liveness verification in different scenarios.

### DETAILED DESCRIPTION OF THE INVENTION

The present invention relates to a method, an apparatus, and a computer program product for biometric identification and/or verification, specifically liveness verification. Biometric identification and verification incorporate techniques of liveness detection or anti-spoofing to detect whether a subject identifying or authenticating with a biometric identifier is a genuine, living being, or a fake representation. In the latter case, a presentation attack, also known as a spoofing attack, may be detected. The subject is usually a human subject.

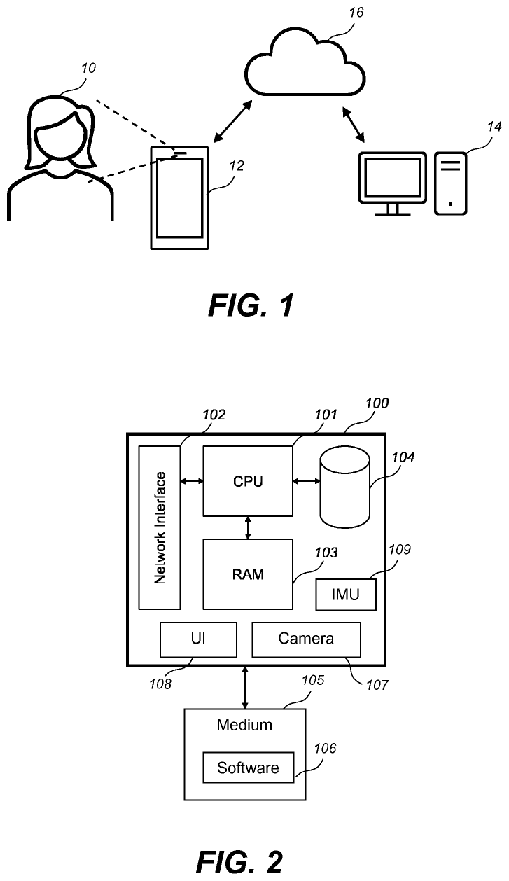

FIG. 1 illustrates an example biometric system. The system comprises a user device 12 and a server 14. The user device 12 is a computing device, and the server is another computing device that is connectable to the user device via a network 16. The user device 12 may be a personal computer, a mobile device, such as a smartphone, tablet computer, laptop, smart watch, or another mobile computing device or wearable device. A user 10 may wish to biometrically identify or verify their identity to perform an action using the user device 12 and/or the server 14, and/or to gain access to an application or data stored in the user device 12 and/or the server 14. Biometric identification and/or verification may be passed using a biometric identifier, also known as a biometric sample, such as the face of the user.

The biometric system of FIG. 1 performs biometric identification and/or verification using the face of the user 10 as the biometric identifier. The user 10 uses a camera of the user device 12 to take a photo or video of their face, and the photo/video is analyzed to determine and/or prove the identity of the user. To prevent unauthorized parties from identifying as the user 10, liveness detection or anti-spoofing is performed to distinguish the living user 10 from a presentation attack.

For example, when the user 10 wishes to access an application on the user device 12 to e.g. sign a document, the biometric identification and/or verification and the liveness detection are performed by the user device 12 on the basis of the data captured by the user device 12. If the identification and/or verification and liveness detection succeed, the user device allows the user 10 to access the application with the user device 12.

In another example, the user wishes to gain access to a building. The user device 12 executing an access control application may send the results of the identification and/or verification and liveness detection to the server 14 executing an access control program, and the server 14 executing the access control program may grant the user 10 access to the building e.g. by sending a command to unlock an electric lock of a door of the building.

In another example, the user wishes to attend an online exam that uses biometric invigilation or proctoring. The user device 12, being e.g. a personal computer or laptop of the user, may send video captured by an integrated or external camera to the server 14. The server 14 may perform the identification and liveness detection, and grant the user access to an exam platform executing on the server 14.

In another example, the user wishes to sign a document using their face as a biometric identifier. The user device may send photo/video data captured by the user device 12 to the server 14. The server 14 may perform the identification and liveness detection, and send the results of the identification and liveness detection to the user device 12. The user device 12 may receive the results and allow the user to sign the document using the user device 12.

FIG. 2 is a schematic diagram depicting an embodiment of an apparatus 100. Preferably, the apparatus is a handheld device, such as the user device 12 of FIG. 1. Alternatively, the apparatus 100 may be a general-purpose computer, such as the server 14 of FIG. 1.

The apparatus 100 includes a central processing unit (CPU) 101. The apparatus may further include a graphics processing unit (GPU) for processing e.g. image and/or video data (not shown). The apparatus 100 includes two memories: a random access memory (RAM) 103 and a non-volatile memory 104. The skilled person understands that a fewer or more memories of various types may be included in the apparatus 100.

The apparatus 100 may be but need not be dedicated hardware. The apparatus may be a virtual machine. The method, described in more detail below, may be executed by the apparatus 100 as a containerized application using operating system (OS)-level virtualization. Alternatively or additionally, the method may be executed in a distributed computing environment and/or as a cloud service.

The apparatus 100 comprises a network interface 102 for communicating with other devices via a network, such as the network 16 of FIG. 1. When the apparatus is e.g. the server 14 of FIG. 1, the apparatus 100 may be located in a data center and accessible via the network 16 of FIG. 1 through the network interface 102. The network interface may comprise one or more network interfaces, such as a cellular network interface, an Internet of Things (IOT) network interface, a personal area network (PAN) interface, and other suitable network interfaces.

The apparatus comprises a camera 107. When the apparatus is a handheld device such as a smartphone, the camera may be the rear camera of the apparatus, or preferably a front camera of the apparatus. The camera 107 is preferably an integral part of the apparatus 100. As an alternative, the camera 107 is an external camera to which the apparatus 100 is connected via e.g. the network 16 of FIG. 1.

The apparatus 100 further comprises a user interface 108. The user interface 108 may comprise various input and/or output devices, such as a display, a touch pad, a touch screen, a speaker, a microphone, and/or a haptic output device. The user interface 108 is preferably an integral part of the apparatus 100. As an alternative, the user interface 108 is an external user interface to which the apparatus 100 is connected via e.g. the network 16 of FIG. 1.

The apparatus 100 further comprises an inertial measurement unit (IMU) 109 comprising one or more accelerometers, gyroscopes, and optionally magnetometers. In the context of the invention, the IMU 109 operates as a movement sensor. Not all sensors of the IMU 109 are necessary for operation as a movement sensor; for example, only the accelerometer(s) may be used. Alternatively, the IMU may be replaced by any movement sensor suitable for measuring biosignals indicative of the heart rate of a subject. The IMU 109 is preferably an integral part of the apparatus 100. In an alternative configuration, the apparatus 100 is connected to the IMU 109 or the alternative movement sensor via e.g. the network 16 of FIG. 1.

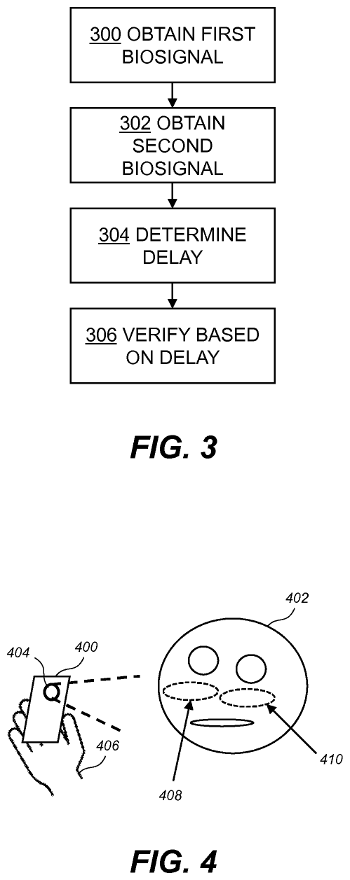

The apparatus 100 is configured to perform the method of FIG. 3 or any of its embodiments. The memories 103, 104 of the apparatus 100 include computer program code that, when executed by the processor of the apparatus, causes the apparatus 100 to perform the method or any of its embodiments. The computer program code 106 may be stored on a (non-transitory) computer-readable medium 105 in the form of a computer program product.

The method of FIG. 3 comprises obtaining 300, 302 first and second biosignals that are indicative of the heart rate of a subject. Referring to FIG. 2, the obtaining may comprise reading the first and second biosignals from the memory, such as the non-volatile memory 104, of the apparatus 100. Referring now to FIG. 1, the user device 12 may measure the first and second biosignals using its sensors, such as the camera 107 and/or the IMU 109 (see FIG. 2). When the apparatus is the server 14 of FIG. 1, the obtaining may comprise receiving the first and second biosignals from the user device 12. The user device may transmit the first and second biosignals to the server 14 e.g., via the network 16 and/or by a network interface of the user device. The server 14 may receive the first and second biosignals via the network 16 and/or by a network interface of the server 14.

The first and second biosignals are time-varying signals whose periodic changes reflect the heart rate of the subject. The first and second biosignals are measured at least partially at the same time to allow comparing them, or to allow comparing at least their parts that overlap in time, with each other. The first and second biosignals are measured by different sensors, which increases the difficulty of carrying out a successful spoofing attack.

The first and second sensors measure biosignals indicative of the heart rate of the subject. The first and second sensors may be of the same type, or they may be of different types. Sensor types include e.g. microphones, cameras, pulse oximeters, pressure sensors, gyroscopes, accelerometers, temperature sensors, radio frequency sensors, and electrodes for measuring bioelectric signals. Radio frequency sensors refer to sensors such as ultra-wideband (UWB) sensors or UWB radars, or Wi-Fi sensors using e.g. the IEEE 802 protocol family. In addition or as an alternative to the above-mentioned sensors, the skilled person may use any sensors that are suitable for measuring biosignals indicative of the heart rate of the subject.

The first and second biosignals may have the same type, or they may have different types. The type of the biosignal refers to the nature of the signal and the phenomena that the signal reflects. Examples of signal types that are indicative of the heart rate of the subject include electrocardiogram (ECG) signals, plethysmography signals such as photoplethysmography (PPG) signals, remote PPG signals, ballistocardiography (BCG) or seismocardiography (SCG) signals, movement sensor signals, and blood pressure signals. In addition or as an alternative to the above-mentioned biosignals, the skilled person may use any biosignals that are indicative of the heart rate of the subject.

To further increase the difficulty of performing a successful spoofing attack, the sensor used to measure the first biosignal is preferably of a different type than the sensor used to measure the second biosignal. Alternatively or additionally, the first biosignal is preferably of a different type than second biosignal. It is more difficult to generate convincing fake signals of different types and/or to mimic different types of sensors at the same time. Most preferably, both the sensor type and the signal type are different between the first and second biosignals.

In the embodiment of FIG. 2, the apparatus 100 uses the camera 107 to obtain the first biosignal. The apparatus 100 captures consecutive or sequential color image data frames that depict the skin, preferably the face, of the subject using the camera 107. The color image data frames may alternatively be in the form of color video data frames. The color image data frames may be extracted by the apparatus 100 from color video data captured by the camera 107. The apparatus 100 acquires the data directly from the camera 107, or from a memory of the apparatus 100 where the camera 107 has stored the data, for example. The apparatus 100 detects color content indicative of the heart rate of the subject in the data and generates the first biosignal based on the detected color content. In this case, the first biosignal is a remote PPG signal.

Changes in the color content of the image/video data originate from pulse and respiration dependent oxygenation changes of blood that circulates in the capillaries close to the skin. The changes may be easiest to detect in locations where there is good blood circulation near the surface of the skin, and the face is such a location.

Changes of color caused by blood pulses take place at slightly different times in different areas of the face. The change is first apparent under the eyes, then on the cheeks, and finally on the forehead. It has been discovered that color differences between different areas of the face and/or changes in the color of the same area over time are indicative of the presence of a heart rate. For example, there may be color changes in patches of skin under the eyes and/or on the cheeks between consecutive images or video frames of a living subject. Alternatively or additionally, there may be color differences between a patch of skin under the eye and a patch of skin on the cheek in the same image or video frame.

The camera 107 may comprise a visible spectrum camera, an infrared scanner, a near-infrared camera, and/or a thermal camera. The camera may be configured to measure, and/or the color video/image data may comprise one or more of: visible spectrum image data, ultraviolet image data, infrared image data, near-infrared image data, and thermal image data. The meaning of the term ‘color’ is herein understood to cover electromagnetic spectra of the light received from the face of the subject also beyond the human visible spectrum. The use of image data beyond the visible electromagnetic spectrum can be used to improve detection of color changes caused by the subject's pulse. Especially the near-infrared image/video data is advantageous for detecting color differences in dark-skinned individuals. As an example, the image/video data may comprise visible spectrum data in red, green, and blue (RGB) channels, and infrared data in an infrared channel. As another example, the blue channel of RBG data may be replaced with the infrared channel such that the image/video comprises visible spectrum data in the red and green channels, and infrared data in the infrared channel. The blue channel contains relatively little relevant information with respect to color changes caused by the pulse and may thus be removed to improve computational efficiency.

The color image/video data may be stored in any suitable format, encoding, and/or color space. The skilled person is free to select any suitable raster image format or video frame format. Example color spaces include red, green, blue (RGB); hue, saturation, intensity (HSI); hue, saturation, value (HSV); hue, saturation, lightness (HSL); and any International Commission on Illumination (CIE) color space such as CIELAB. Other suitable color spaces may be used as well. The data may be converted from a first color space to a second color space, such as from RGB to CIELAB to enhance detectable color changes.

The apparatus 100 may detect the color content by identifying one or more skin regions in each of a plurality of color video data frames of the color video data, or alternatively in each of a plurality of consecutive color image data frames of the color image data. The plurality of skin regions may be predetermined, and the predetermined skin regions may be stored in the memory of the apparatus 100, for example. The identifying may comprise tracking the face of the subject and/or identifying locations of one or more anatomical features or landmarks on the face of the subject. The landmarks may represent the eyebrows, eyes, nose, lips, and/or jawline of the subject. Face tracking and identification of landmarks are generally known in the art and disclosed e.g., in “Real-time face alignment: evaluation methods, training strategies and implementation optimization”, a Master's thesis by Constantino Álvarez Casado, published on 2020Dec. 18. The plurality of skin regions may be identified on the basis of the identified locations of the landmarks. For example, the skin regions may be bounded by specific landmarks, and/or defined by predetermined distances from the landmarks. As an example, a forehead skin region may be bounded by hairline landmarks and eyebrow landmarks. As another example, an under-eye region may cover a predetermined distance downwards from eye landmarks.

A plurality of skin regions may together form a composite skin region. For example, the left and right forehead skin regions may together form a forehead skin region. The forehead skin region may then be considered as one skin region. Composite skin regions may provide benefits in relation to how the skin regions are identified. For example, it may be computationally more accurate and/or efficient to identify two or more parts of a skin region separately, e.g., based on the landmarks of the subject's face, and then join them together.

The apparatus 100 may extract a skin region data set from each one of the one or more identified skin regions in each of the plurality of color video/image data frames. Each skin region data set contains data of the image/video frame that depicts the skin region in the data frame. The extracting may be performed using (bit) mask(s) and/or array/matrix indexing. The extracting may be performed in-place, i.e., the locations of the skin region data sets are identified in the data frame(s), and subsequent processing of the skin region data sets is performed directly on the data of the data frame(s). Alternatively, or additionally, the skin region data sets may be excerpted from the data frame(s) e.g., by a copy operation, and subsequent processing of the skin region data sets is performed on the excerpted skin region data sets.

The apparatus 100 may detect the color content of each one of the extracted the skin region data sets, wherein the color content is indicative of the heart rate of the subject. The apparatus 100 may generate the first biosignal based on the detected color contents e.g. as a sequence of the color contents of consecutive image/video frames. Separate signals may be generated for different skin regions, if more than one skin region is used.

To further improve the verification accuracy, the first biosignal may comprise color distributions of one or more skin regions of the subject's face. Color distributions characterize the color content of a skin region with minimal loss of information, when compared to e.g., averaging of color values. A distribution type of the color distributions may be a probability density function, cumulative distribution function, probability distribution, histogram, local binary pattern histogram, or co-occurrence matrix, of pixels or data values of the respective skin region data set, for example.

The selection of the distribution type and how they are computed may depend on the color space of the color image/video data frames and correspondingly the color space of the skin region data sets. As color image/video data usually contains multiple channels, such as the red, green and blue channels in RGB data, or the hue, saturation, value channels in HSV data, the color distributions may be multivariate distributions. For example, a multivariate probability density function may be computed for RGB data of a skin region data set. The (three) variables of the distribution are in this case the red, green and blue channels of the RGB data.

The apparatus 100 may compute a plurality of color distributions, each color distribution being computed on the basis of one of the plurality of skin region data sets. The apparatus 100 may further detect the color the color content of each extracted skin region data set based on the color distribution computed on the basis of the skin region data set in question. The apparatus may further generate the first biosignal based on the detected color contents, e.g. as a sequence of the color distributions. Separate signals may be generated for different skin regions, if more than one skin region is used. The resulting first biosignal may still be considered a remote PPG signal, or a refined or processed remote PPG signal.

Further details related to processing facial color changes and color distributions are available in the applicant's pending Finnish patent application 20225646, filed on Aug. 7, 2022, which is incorporated by reference herein in its entirety.

In the embodiment of FIG. 2, the apparatus 100 uses the IMU 109 to obtain the second biosignal. The IMU 109, or at least its accelerometer(s), acts as a movement sensor and measures movement data of the subject. The heart rate of the subject causes minute periodic movement of the subject, resulting in a BCG signal measured by the IMU 109 or its accelerometer(s).

When the apparatus 100 is a hand-held device, such as a smartphone, an example solution for measuring a signal indicative of the subject's heart rate using smartphone accelerometer data can be found from K. Jiokeng, G. Jakllari and A.-L. Beylot, “HandRate: Heart Rate Monitoring While Simply Holding a Smartphone,” 2021 IEEE International Conference on Pervasive Computing and Communications (PerCom), Kassel, Germany, 2021, pp. 1-11, doi: 10.1109/PERCOM50583.2021.9439134. Further example solutions for measuring signals indicative of the subject's heart rate using smartphone accelerometers can be found from: Federica Landreani & Enrico Gianluca Caiani (2017) Smartphone accelerometers for the detection of heart rate, Expert Review of Medical Devices, 14:12, 935-948, DOI: 10.1080/17434440.2017.1407647. When the apparatus 100 is a wearable device, details of processing wearable accelerometer data to detect a heart rate are provided in M. Haescher et al., “Seismotracker: Upgrade any smart wearable to enable a sensing of heart rate, respiration rate, and microvibrations,” in Proc. 2016 CHI Conf. Extended Abstr. Hum. Factors Comput. Syst., J. Kaye et al. Eds., New York, USA: ACM Press, 2016, pp. 2209-2216. The applicant has found that although not recommended in FIG. 4 of Haescher et al., the methods of Hasecher et al. may also be applied to acceleration signals that are obtained from the subject's hand.

The movement data and thus the resulting BCG signal may be measured from any part of the subject's body, but preferably from a different location than where the first biosignal was measured. Example locations for measuring the movement data include the subject's face, hand, finger, wrist, and (inner) elbow. Preferably, when the first biosignal is measured from a first body part of te subject, the second biosignal is measured from a different body part of the subject. When the first biosignal is measured from the face of the subject, the second biosignal is preferably measured from the hand of the subject. When the first and second biosignals are measured from different body parts, possibly predetermined different body parts, the difficulty of carrying out a successful spoofing attack is increased as biosignals from two different origins would need to be successfully mimicked.

The first and second biosignals may be measured from body parts with different distances to the heart. The face and hand of the subject are such body parts. Different distances to the heart result in different pulse travel times to the body parts, which the skilled person can account for when determining expected delays as explained in more detail later.

FIG. 4 illustrates a scenario of using a second embodiment of the apparatus 400 according to the invention. The apparatus 400 is similar to the apparatus shown and described in relation to FIG. 2. Additionally, the apparatus 400 is a hand-held device, which allows for achieving further advantages. The subject 402 operates the hand-held apparatus 400 by holding it in their hand. As described in relation to FIG. 2, the apparatus 400 measures the first biosignal from skin regions 408, 410 of the subject's face by capturing image or video data that depicts the face of the subject using the front camera 404 of the apparatus 400. The apparatus 400 measures the second biosignal by measuring movement of the subject's hand 406 used to hold the apparatus 400 using a movement sensor of the apparatus 400. The apparatus 400 outputs instructions to the subject 402 via its user interface, e.g. via a display of the apparatus 400. The instructions guide the subject 402 to hold the apparatus still in the subject's hand, and to position the apparatus 400 so that the subject's face is centered in the view of the front camera 404. The instructions may guide the subject to rest their elbow on a stationary object to provide a standardized measurement scenario. The apparatus 400 displays the view of the front camera 404 to the subject 402 on the display of the apparatus 400. The apparatus 400 determines the time during which the subject's face is within a predetermined region of the front camera's field of view. During this time, the apparatus 400 measures the first and second biosignals. The predetermined region may have an oval shape. The apparatus 400 determines when the length of time during which the subject's face has been within the predetermined region of the front camera's field of view exceeds a predetermined threshold that corresponds to a sufficient length of the first and second biosignals. When the length of time exceeds the threshold, the apparatus 400 outputs a success indication to the subject 402 via its user interface.

Referring again to FIG. 3, the method further comprises determining 304 a delay between the first biosignal and the second biosignal. Whether the delay is determined from the first biosignal to the second biosignal or vice versa is not significant, as long as the same approach is used consistently. Herein, the delay is determined from the first biosignal to the second biosignal such that a positive delay corresponds to the second biosignal lagging behind the first biosignal. The skilled person is aware of various techniques for determining the delay, and any suitable technique can be used. For example, time domain and/or frequency domain techniques may be used. Both the apparatus 100 of FIG. 2 and the apparatus 400 of FIG. 4 may determine the delay in the same manner.

In the time domain, the delay may be determined as a time difference between events in the first and second biosignals. The apparatus 100, 400 detects a first event in the first biosignal, and a second event in the second biosignal. The first and second events are corresponding events that have a predetermined expected delay. The first and second events may be e.g. peaks and/or troughs in PPG, ECG, BCG, blood pressure, or movement sensor signals, for example. For ECG signals, the event may be one or more of the P, Q, R, S, or T waves, and/or the QRS complex, for example.

In the frequency domain, the delay may be determined as a phase difference between the first and second biosignals. Phase information of the first and second biosignals may be obtained e.g. by applying a short-time fast Fourier transformation (STFFT) to the first biosignal and to the second biosignal. Correlation-based approaches known to the skilled person may also be used to obtain the phase difference between the first and second biosignals.

The length of the first and second biosignals needed for determining the delay depends on the technique used for determining the delay. Preferably the first and second biosignals have a duration of at least three heartbeats, and thus the length of the signals may depend on the heart rate of the subject. Typically, aiming a front camera of a hand-held device to take a photo or video as shown in the scenario of FIG. 4 can take up to 10 seconds and thus include approximately 10 to 15 heartbeats, and first and second biosignals measured for this duration provide sufficiently long signals for determining the delay.

Referring again to FIG. 3, whether the subject is alive or not is verified 306 based on the determined delay between the first and second biosignals. The apparatus 100, 400 (see FIG. 2 and FIG. 4) performs the verification by comparing the determined delay to an expected delay. If the determined delay matches the expected delay, the apparatus 100, 400 determines the result of the liveness verification to be a pass. Otherwise, the apparatus 100, 400 determines the result to be a failure. The expected delay may be stored in the memory of the apparatus as a fixed value, possibly including an error margin, or preferably as a range. Alternatively or additionally, the expected delay may be programmatically determined by the apparatus as part of the method.

When expected dela is a fixed value, the determined delay matching the expected delay means that the determined delay corresponds to the expected delay value, e.g. a determined delay of 200 ms matches an expected delay of 200 ms. When an error margin is included in the expected delay, the error margin is considered in the comparing: for example, a determined delay of 180 ms matches an expected delay of 200 ms ±20 ms. When the expected delay is a range, the determined delay matches the expected delay if it falls within the range. For example, a determined delay of 180 ms matches an expected delay of 180 to 220 ms.

The determined delay preferably comprises a time-varying delay signal comprising a plurality of delay values. Each delay value may represent the delay of a single heartbeat between the first biosignal and the second biosignal. The delay signal represents the delay between the first and second biosignals over the duration of the two biosignals. Such a delay signal may be obtained e.g. using the STFFT approach described above (frequency domain), or by determining the time differences between each pair of corresponding events in the first and second biosignals (time domain). Each value of the delay signal may be compared to the expected delay by the apparatus 100, 400 to determine that the values of the delay signal match the expected delay for the entire duration of the delay signal. If all values of the delay signal match the expected delay, the apparatus 100, 400 determines the result of the liveness verification to be a pass. Otherwise, if all values of the delay signal do not match the expected delay, the apparatus 100, 400 determines that the liveness verification result is a failure. This further increases sensitivity of the liveness verification as changes in the delay over the measurement period are accounted for. Further, use of the time-varying delay signal allows for considering natural variations in heart rate over time or between heart beats—and difference in such variations between the first and second biosignals may indicate a spoofing attempt that is detected by the apparatus based on the delays.

The expected delay depends on the technical details of the biometric system and its components. The first and second biosignals are preferably measured using the same device to simplify the resulting delays and to reduce the risk of malicious data injection to separate measurement devices. The same device is further preferably used to perform the processing of the measured biosignals, i.e. the method described herein, for the same reasons.

For example, in the scenario of FIG. 4, measurements performed by the applicant indicate that the expected delay is in the range of 140 to 200 milliseconds (ms) when the apparatus 400 is a typical smartphone. The delay is from the face to the hand, i.e. the second biosignal is expected to lag behind the first biosignal by approximately 140 to 200 ms. This delay includes a delay caused by the frame rate of the front camera, which is in the range of 20 to 30 ms, and the physiological delay between pulse travel times to the face and the hand. Blood pressure is known to affect pulse travel times, with a high blood pressure decreasing the travel time and thus the delay as well. The delay does not include the time for processing the biosignals, such as determining the delay and verifying liveness of the subject. The skilled person is able to use his common general knowledge and routine measurements to estimate the physiological delays caused by different pulse travel times, and the effects of blood pressures on the delays, as well as the delays caused by the signal measurements themselves.

Correspondingly, the expected delay accounts for delays of both technological origin (such as image capture times, e.g. 16.7 ms at 60 frames per second (fps) or 20 to 30 ms as above) and of biological origin (e.g. pulse transmission time differences). The expected delay is small enough that phase wrap-around is not expected. As mentioned above, the skilled person is able to determine and verify the expected delay for any particular solution by routine measurements.

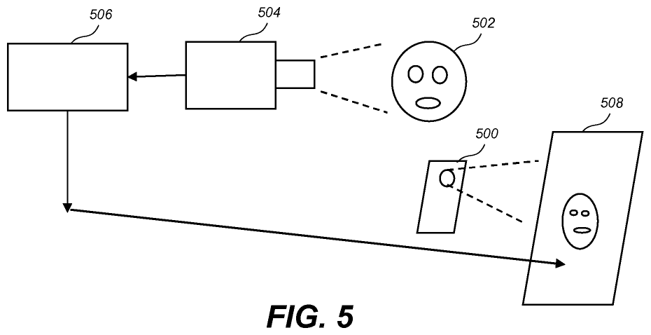

FIG. 5 shows a scenario where a face-swapping deepfake video is created with highly optimized computing technology. The apparatus 500 is a smartphone that otherwise corresponds to the apparatuses 100, 400 of FIG. 2 and FIG. 4. An impostor 502 presents himself as the victim of the attack by converting a video captured by a first camera 504 to a video that simulates the appearance of the victim using a computer 506. The computer 506 displays the converted video on a display 508. The apparatus 500 is held in the hand of the impostor or their assistant, and the camera of the apparatus 500 is aimed at the display 508. The apparatus 500 obtains the first and second biosignals from the face shown on the display 508 and from the hand holding the apparatus 500 as described above.

Using an efficient GPU, the additional delay between the first and second biosignals is expected to be several minutes. With a modern supercomputer, the delay is still expected to be several seconds. Using highly optimized theoretical computing technology, the total delay from the impostor 502 to the apparatus 500 is still evaluated to be over 70 ms: 16.7 ms for camera integration time when images are captured by the first camera 504 at 60 fps, 16.7 ms transfer time to computer at 60 Hz, 16.7 ms for real-time computing time for producing the fake video at 60 Hz, 16.7 ms transfer time to the display 508 at 60 Hz, and 5 ms of display lag. The resulting delay is significant enough to be caught in the liveness verification performed by the apparatus 500, as the delay between the first biosignal from the camera of the apparatus 500 and the second biosignal measured by the IMU does not match the expected delay. As the signal from the impostor's 502 face is delayed due to processing by the first camera 504, the computer 506, and the display 508, the delay between the first and second biosignals becomes too short. With less advanced technology, the deviation from the expected delay is even larger.

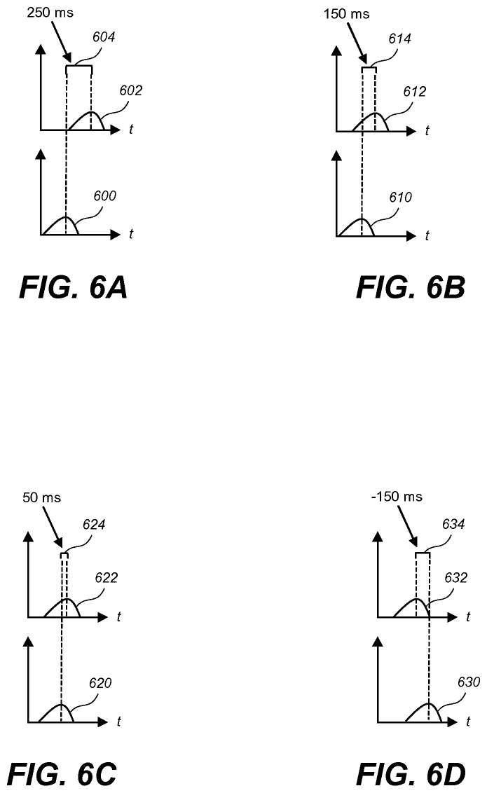

FIG. 6A-6D illustrate liveness verification scenarios for different delays, with an expected delay of 140 to 200 ms as described above. The figures depict the first and second biosignals in the time domain, but the method used to evaluate the delay does not need to be performed in the time domain—it may, for example, be performed in the frequency domain. The delays between the first and second biosignals are illustrated using the times at which corresponding events, depicted as signal peaks, occur in both biosignals.

In FIG. 6A, there is a delay 604 of 250 ms between the first biosignal 600 and the second biosignal 602. This delay is too large to match the expected delay of 140 to 200 ms, so the liveness verification is failed.

In FIG. 6B, there is a delay 614 of 150 ms between the first biosignal 610 and the second biosignal 612. This delay matches the expected delay of 140 to 200 ms, so the liveness verification is passed. This scenario corresponds to a genuine subject using the biometric system.

In FIG. 6C, there is a delay 624 of 50 ms between the first biosignal 620 and the second biosignal 622. This delay is too small match the expected delay of 140 to 200 ms, so the liveness verification is failed. The scenario of FIG. 6C may correspond to the scenario of FIG. 5, for example. Such a scenario may occur when an artificial signal is generated based on the second biosignal and injected into the first biosignal. For example, in the scenario of FIG. 5, artificial color changes can be generated based on the second biosignal obtained from the impostor's hand, and injected into the video shown on the screen 508

In FIG. 6D, there is a delay 634 of −150 ms between the first biosignal 630 and the second biosignal 632. The magnitude of the delay corresponds to that in FIG. 6B, but the first and second biosignals are in an incorrect order. This delay does not match the expected delay of 140 to 200 ms, so the liveness verification is failed. The scenario of FIG. 6D may correspond to the scenario of FIG. 5, similarly to that of FIG. 6C, but with an even longer delay in the first biosignal.

Referring again to FIG. 1, FIG. 2 and FIG. 4, the apparatus 100, 400 is further configured to output a pass result or a failure result of the verification. The outputting may comprise writing the verification result to a memory, such as the non-volatile memory 104 of the apparatus 100 (see FIG. 2). Alternatively, or additionally, the outputting may comprise transmitting the verification result e.g., via the network 16 (see FIG. 1) and/or by the network interface 102 (see FIG. 2). For example, when the method is performed by the user device 12 (see FIG. 1), the user device 12 may transmit the verification result to the server 14 via the network 16 using a network interface of the user device. The server 14 may receive the detected liveness via the network 16 and/or by a network interface of the server 14. Alternatively, or additionally, the outputting may comprise outputting the verification result by the user interface 108 (see FIG. 2) of the apparatus 100.

When the verification result is a pass, the output verification result may be used by a further computer program or module to authorize, authenticate, or grant the subject access to perform further steps. When the verification result is a failure, the output verification result may respectively be used to prevent access, authorization and/or authentication in view of a likely presentation attack.

If desired, the different functions discussed herein may be performed in a different order and/or concurrently with other. The embodiments and features, if any, described in this specification that do not fall under the scope of the independent claims are to be interpreted as examples useful for understanding various embodiments of the invention.

1. A method for biometric verification, the method comprising:
Obtaining a first biosignal indicative of a heart rate of a subject, wherein the first biosignal has been measured by a first sensor;
Obtaining a second biosignal indicative of the heart rate of the subject, wherein the second biosignal has been measured by a second sensor that is different from the first sensor, and wherein the first and second biosignals have been measured at the same time;
determining a delay between the first and second biosignals; and
verifying liveness of the subject based on the determined delay.

2. (canceled)

3. The method of claim 1, wherein verifying the liveness of the subject comprises comparing the determined delay to an expected delay, and, if the determined delay matches the expected delay, passing liveness verification of the subject, and otherwise failing the liveness verification of the subject.

4. The method of claim 3, wherein the method comprises determining a delay signal comprising a plurality of delay values between the first and second biosignals, and wherein each value of the delay signal is compared to the expected delay, and, if all values of the delay signal match the expected delay, passing liveness verification of the subject, and otherwise failing the liveness verification of the subject.

5. The method of claim 4, wherein each delay value of the delay signal corresponds to a delay of a single heartbeat between the first biosignal and the second biosignal.

6. The method of claim 1, wherein the first sensor is of a different type than the second sensor.

7. The method of any preceding claim 1, wherein the first biosignal is of a different type than the second biosignal.

8. The method of claim 1, wherein the first biosignal comprises a remote photoplethysmography signal, and the second biosignal comprises a ballistocardiography signal.

9. The method of claim 1, wherein the first biosignal comprises a remote photoplethysmography signal comprising color distributions of one or more skin regions of the face of the subject.

10. The method of claim 1, wherein the first biosignal and the second biosignal are measured from different body parts of the subject.

11. The method of claim 1, wherein the first biosignal is measured from a first body part having a first distance to the heart of the subject, and the second biosignal is measured from a second body part having a second distance to the heart of the subject, wherein the first and second distances are different.

12. The method of claim 1, wherein the first biosignal is measured from the face of the subject, and the second biosignal is measured from the hand of the subject.

13. (canceled)

14. The method of claim 1, wherein the first sensor comprises a camera configured to measure image data indicative of the heart rate of the subject.

15. The method of claim 14, wherein obtaining the first biosignal comprises:
acquiring a plurality of color image data frames depicting the face of the subject;
detecting color content indicative of the heart rate of the subject in the plurality of color image data frames; and
generating the first biosignal based on the detected color content.

16. The method of claim 15, wherein detecting the color content comprises:
identifying one or more skin regions in each of the plurality of color image data frames;
extracting a skin region data set from each of the one or more identified skin regions in each of the plurality of color image data frames; and
detecting the color content of each extracted skin region data set.

17. The method of claim 16, wherein the method further comprises:
computing a plurality of color distributions, each color distribution being computed on the basis of one of the plurality of skin region data sets; and
detecting the color content of each extracted skin region data set based on the color distribution computed on the basis of said skin region data set.

18. (canceled)

19. The method of claim 1, wherein the second sensor comprises a movement sensor configured to measure movement sensor data indicative of the heart rate of the subject.

20. The method of claim 19, wherein the second biosignal comprises a ballistocardiography signal based on the movement sensor data.

21. The method of claim 1, wherein the method further comprises measuring the first and second biosignals using the same device.

22. An apparatus comprising at least one processor, at least one memory including computer program code, the at least one memory and the computer program code configured to, with the at least one processor, cause the apparatus to perform:
obtaining a first biosignal indicative of a heart rate of a subject, wherein the first biosignal has been measured by a first sensor;
obtaining a second biosignal indicative of the heart rate of the subject, wherein the second biosignal has been measured by a second sensor that is different from the first sensor, and wherein the first and second biosignals have been measured at the same time;
determining a delay between the first and second biosignals; and
verifying liveness of the subject based on the determined delay

23-25. (canceled)

26. A non-transitory computer-readable medium comprising computer program code configured to, when executed by at least one processor, cause an apparatus to perform:
obtaining a first biosignal indicative of a heart rate of a subject, wherein the first biosignal has been measured by a first sensor;
obtaining a second biosignal indicative of the heart rate of the subject, wherein the second biosignal has been measured by a second sensor that is different from the first sensor, and wherein the first and second biosignals have been measured at the same time;
determining a delay between the first and second biosignals; and
verifying liveness of the subject based on the determined delay.
